(19) United States
(12) Patent Application Publication
(10) Pub. No.: US 2026/0289484 A1
(43) Pub. Date: Sep. 24, 2026

(54) INTELLIGENT SELECTION METHOD AND SYSTEM FOR PACKAGING MATERIAL BASED ON MULTIDIMENSIONAL DATA ANALYSIS

(71) Applicant: Gutelaifu Intelligent Technology (Shenzhen) Co., Ltd., Shenzhen (CN)

(72) Inventor: Shenggan ZHU, Shenzhen (CN)

(21) Appl. No.: 19/565,229

(22) Filed: Mar. 12, 2026

(30) Foreign Application Priority Data
Mar. 24, 2025 (CN) 202510348567.6

(51) Int. Cl. G06Q 10/0832

(52) U.S. Cl. CPC G06Q 10/0832

(57) ABSTRACT

The present invention provides an intelligent selection method and system for a packaging material based on multidimensional data analysis. An environmental sensing module acquires vibration, deformation, and temperature and humidity data in real time to construct a feature matrix. A data analysis module integrates a historical transportation case database and logistics task duration to predict material life and associates supply chain inventory to generate remaining life assessment. A decision-making engine module dynamically allocates weights for costs, safety, and environmental protection, and generates a multi-level decision-making solution in combination with supplier credit scores. An execution control module verifies a material dimensional tolerance and drives replacement, triggering model calibration through a 5% deviation threshold. The system is equipped with a grading standard for a humidity-sensitive material and a deformation safety limit.

### CROSS-REFERENCE TO RELATED APPLICATIONS

The present application claims priority to Chinese patent application No. 202510348567.6, titled “INTELLIGENT SELECTION METHOD AND SYSTEM FOR PACKAGING MATERIAL BASED ON MULTIDIMENSIONAL DATA ANALYSIS”, filed on 24 Mar. 2025, the entire contents of which are incorporated herein by reference.

### FIELD OF TECHNOLOGY

The present invention relates to the field of material management, and in particular to an intelligent selection method and system for a packaging material based on multidimensional data analysis.

### BACKGROUND

Modern logistics systems impose higher requirements on packaging material performance; the globalization of goods circulation increases the complexity of transportation environments; conventional material selection methods rely on manual experience and static parameters; vibration and variations in temperature and humidity during transportation lead to dynamic degradation of material performance; upgraded environmental protection regulations require balancing material costs and sustainability; and intelligent decision-making systems have become a key direction for industry advancement.

In current mainstream solutions, a single environmental sensor is used to monitor transportation conditions; a static selection model is built based on a material hardness and thickness; historical transportation data is introduced for some solutions to construct a linear prediction formula; a supplier database is integrated for inventory matching in few of the solutions; a fixed-weight algorithm is used to balance costs and safety indicators; and a manual intervention process is triggered through a threshold-based alarm mechanism.

However, the existing technologies suffer from the following deficiencies: a lack of a multi-source data fusion and analysis capability; limited monitoring dimensions of environmental parameters, resulting in prediction deviations; static models that fail to reflect dynamic degradation rules of material performance; inventory matching that ignores supplier capacity fluctuations and logistics timeliness; fixed-weight algorithms that are difficult to adapt to changing requirements at different transportation stages; and delayed manual intervention responses, thereby leading to a risk of delayed material replacement.

### SUMMARY

### (I) Technical Problems to Be Solved

In view of the deficiencies of the prior art, the present invention provides an intelligent selection method and system for a packaging material based on multidimensional data analysis, where the problems to be solved in the background include: a lack of a multi-source data fusion and analysis capability; limited monitoring dimensions of environmental parameters, resulting in prediction deviations; static models that fail to reflect dynamic degradation rules of material performance; inventory matching that ignores supplier capacity fluctuations and logistics timeliness; fixed-weight algorithms that are difficult to adapt to changing requirements at different transportation stages; and delayed manual intervention responses, thereby leading to a risk of delayed material replacement.

### (II) Technical solution

To achieve the above objectives, the present invention is implemented through the following technical solution: an intelligent selection method and system for a packaging material based on multidimensional data analysis, including an environmental sensing module, a data analysis module, a decision-making engine module, and an execution control module, where
the environmental sensing module acquires vibration spectrum, surface strain, and temperature-humidity data in real time via a sensor network deployed on a surface of the packaging box, and generates an environmental state matrix after feature extraction, where the sensor network includes an automatic calibration unit;
the data analysis module is internally provided with a historical transportation case database that stores performance degradation records of different materials in a transportation environment, establishes, upon receiving the environmental state matrix, a correlation model between vibration energy distribution and material performance degradation, and predicts remaining material life in conjunction with inventory turnover data from a supply chain database, and establishes a data interface with a logistics management system to obtain transportation task duration and route planning data in real time;
the decision-making engine module dynamically adjusts priorities of costs, safety, and an environmental protection objective based on transportation phases, integrates a material life prediction result with supplier capacity data, and generates a multi-level decision-making tree including an automatic execution solution and a contingency plan;
the execution control module: pre-stores a packaging box dimension tolerance standard, a structural deformation safety limit, and a registered dimension database; converts a selected solution into a material replacement instruction, drives an actuating mechanism to complete the operation, acquires an error between actual transportation data and a predicted value, and feeds back the error to the data analysis module to trigger dynamic model parameter calibration, and the predicted error of the model includes three types: an environmental sensing error, a material performance error, and an execution control error; and
the supply chain database includes a material moisture resistance characteristic index table and a critical safety threshold database for hygroscopic expansion coefficients, records a deformation coefficient, a protection level, and an irreversible deformation threshold of each material under different humidity environments, and marks a humidity sensitivity level; and performs dynamic calculation based on a delivery punctuality rate and a quality inspection pass rate to establish a supplier credit score system, and sets a credit score threshold to a value of a normal distribution curve of historical fulfillment data.

Preferably, the environmental sensing module acquires transportation environment data via a sensor network deployed on all six surfaces of the packaging box; the sensor network includes three types of detection units: a vibration sensor, a deformation sensor, and a temperature-humidity sensor; the vibration sensor acquires three-dimensional acceleration data at a 1 kHz sampling rate and extracts energy distribution features in a frequency band of 0-500 Hz via fast Fourier transform. The deformation sensor monitors surface microstrain at a frequency of 100 Hz and uses a spatial interpolation algorithm to build a deformation gradient field model. The temperature-humidity sensor synchronously detects environmental parameters on each surface, and triggers an abnormal flag when a temperature difference between adjacent surfaces exceeds 3° C. or a humidity difference between adjacent surfaces exceeds 15% RH. All sensor data are transmitted via a low-power wireless network to an edge node, where timestamp alignment and standardization are performed to generate an environmental state matrix including frequency-domain energy spectra, deformation gradients, and temperature-humidity distributions.

Preferably, the data analysis module: is equipped with a historical transportation case database, and stores records of material performance degradation under coupled effects of vibration, temperature, and humidity; after receiving the environmental state matrix, extracts a dominant vibration frequency energy proportion feature and performs matching degree analysis with a material natural frequency database; initiates a real-time re-evaluation process of material cushioning efficiency when an energy proportion within a resonance frequency band exceeds a preset threshold; and calculates a confidence interval for remaining material life combined with inventory turnover data from the supply chain database; and triggers a real-time inventory verification process if predicted life is shorter than transportation task duration obtained from the logistics management system.

Preferably, the decision-making engine module dynamically adjusts target function weights based on transportation phases: in a preparation phase, a cost weight is set to 0.6, a safety weight is set to 0.3, and an environmental protection weight is set to 0.1; in a transportation phase, the safety weight is increased to 0.5; and in a customs clearance stage, an environmental compliance constraint is introduced, to apply a gain coefficient of 1.2 to an environmental objective. The decision-making engine module integrates a material life prediction result with real-time supplier capacity data to generate a three-tier recommendation solution. When inventory is less than 120% of a current transportation demand, a priority of the material is automatically lowered, and a supplier database is linked to select a list of alternative suppliers with sufficient capacity and logistics timeliness that satisfy a shortest delivery cycle.

Preferably, the execution control module pre-stores a packaging box dimension tolerance standard and a structural deformation safety limit, receives material specification parameters from the decision-making engine module, matches material dimensions with a packaging box 3D model in a registered dimension database, and triggers a solution regeneration process based on actual dimension constraints if a tolerance exceeds a preset safety range; and drives a pneumatic-electromagnetic hybrid actuating mechanism to complete material replacement, receives real-time positioning accuracy feedback from a displacement sensor, initiates a PID dynamic correction mechanism if a measured position deviation exceeds 0.5 mm, and, after operation completion, acquires actual transportation data and calculates a deviation from a predicted value and feeds the deviation to the data analysis module.

Preferably, the supply chain database includes a material moisture resistance characteristic index table and a supplier credit score system. The moisture resistance index table records deformation coefficients and protection grades of respective materials within a humidity range of 30%-90% RH, and materials having a hygroscopic expansion coefficient≥of 0.5 mm/% RH are designated as humidity-sensitive materials. The credit score system dynamically calculates supplier scores based on a delivery punctuality rate weight and a quality inspection pass rate weight, the delivery punctuality rate weight being 0.6 and the quality inspection pass rate weight being 0.4. A credit score threshold is defined as a μ−2σ value of a normal distribution of historical fulfillment data. When a supplier score falls below the threshold, a material option provided by the supplier is automatically excluded. Actual loss rate data fed back by the execution control module is incorporated into a subsequent scoring cycle with a weight of 0.2.

Preferably, when vibration spectrum analysis identifies that an energy value in an ±10% frequency band of a material's intrinsic frequency±increases by more than 50% over a baseline within 2 seconds, the environmental sensing module sends a resonance warning signal to the data analysis module, the data analysis module retrieves transportation records with the same frequency band feature from a historical transportation case database, calculates a damage rate of a corresponding material, and sends a solution re-evaluation request to the decision-making engine module if the damage rate exceeds a safety threshold, and the decision-making engine module generates a list of alternative material priorities and links the supplier database to select an alternative supplier that satisfies delivery timeliness.

Preferably, a handling strategy for a humidity-sensitive material implemented by the data analysis module includes the following steps: retrieving, from the supply chain database, a list of materials having a hygroscopic expansion coefficient≥of 0.5 mm/% RH; upon detecting a humidity change rate>of 15% RH/h in environmental sensing data, initiating a dedicated prediction model, where the prediction model employs an LSTM neural network, an input layer of which includes a current humidity value, a humidity change gradient, and a material hygroscopic curve feature, and an output layer of which predicts a deformation amount within subsequent two hours; and triggering a three-level response mechanism when a predicted deformation exceeds 80% of a structural deformation safety limit: a downgraded use recommendation is sent to the decision-making engine module in a first level; an automatic desiccant dispensing system is activated in a second level; a moisture-resistant material replacement solution is invoked in a third level; and an abnormal data packet is labeled as high-value training sample and is preferentially used for an iterative model update.

Preferably, a fast approval process of the execution control module includes the following steps: when the decision-making engine module generates a recommended list of alternative materials, the system automatically retrieves a customs HS code database to verify material compliance and adds an electronic geofencing mark to a solution involving a restricted substance; an approval request is pushed to a responsible engineer via an enterprise WeChat API, with a material parameter comparison table and a risk analysis report attached; the engineer performs confirmation by means of an electronic signature using a digital certificate, and the system automatically records a signature timestamp and a device fingerprint; upon approval, a production work order is immediately issued to an MES system, and a WMS inventory state is synchronously updated; the entire process is required to be completed within 15 minutes, and if approval is not completed within the time limit, the process is automatically escalated to a superior supervisor and a standby contingency plan is initiated.

Preferably, a dynamic model parameter calibration process includes the following operations: upon receiving a calibration request from the execution control module, raw sensor data within a corresponding time window is extracted from a data lake; vibration signals are subjected to joint time-frequency analysis, and instantaneous frequency features are extracted using a Wigner-Ville distribution; actual material loss data is compared with predicted values by residual analysis to identify primary error contribution dimensions; for a humidity-sensitive material, Bayesian optimization is preferentially employed to update a humidity coupling coefficient in a degradation model, and a Jacobian matrix is computed in each iteration to determine a parameter adjustment direction; and upon completion of calibration, a model file carrying a version identifier is generated and, after verification by a digital signature, is released to the decision-making engine and a production database.

Preferably, the data analysis module periodically classifies and attributes model prediction errors. At the end of each quarter, a global health assessment is initiated, and a model prediction error is classified by source. When an environmental sensing error exceeds 5%, channel quality analysis is performed on the sensor network, and node distribution is optimized. When a material performance error exceeds 8%, supplier-provided material samples are resampled for destructive tests, and a base parameter database is updated. When an execution control error exceeds 2 mm, laser interferometer precision measurement is performed on a transmission component of a driving mechanism, and a ball screw whose wear exceeds a tolerance band is replaced. All optimization operations are performed as a closed-loop control, and each adjustment needs to undergo validation testing across three complete transportation cycles to ensure that system stability improvement satisfies a preset KPI target.

Preferably, after installation of a new material is completed, the execution control module monitors an initial cushioning efficiency parameter in real time; when a deviation between a measured value and a model-predicted value exceeds 5%, the execution control module sends a calibration request to the data analysis module, the calibration request including an environmental data timestamp and a material batch code; the data analysis module extracts environmental feature data corresponding to a relevant time period and preferentially updates a performance degradation model for the humidity-sensitive material; and an updated model parameter is synchronously delivered to the decision-making engine module after verification by a digital signature.

Preferably, a registered dimension database for the packaging box includes the following: performing full-dimension measurements on each batch of packaging boxes using a three-dimensional laser scanner to acquire spatial coordinates of no fewer than 2,000 feature points; employing an ICP algorithm to register point cloud data with design drawings and to calculate statistical distribution characteristics of respective dimensional parameters; setting dynamic tolerance bands for a length, width, and height, a width of each tolerance band being defined as ±(0.1%×of a nominal value+0.5 mm); when a standard deviation of dimensions of packaging boxes within a same batch exceeds 50% of a corresponding tolerance band, automatically generating a quality anomaly report and triggering a supplier deduction procedure; and performing database defragmentation and index rebuilding on a monthly basis to ensure query response time of less than 50 ms.

### (III) Beneficial Effects

The present invention provides an intelligent selection method and system for a packaging material based on multidimensional data analysis. The present invention has the following beneficial effects.
1. According to the present invention, the accuracy and adaptability of packaging material selection are significantly enhanced through multi-source data fusion and a dynamic decision-making mechanism. The environmental sensing module captures the vibration spectra, surface strain, and temperature-humidity gradient data in real time, and establishes the material performance degradation prediction model in combination with the historical transportation case database to effectively identify a resonance risk and a critical deformation state, thereby ensuring transportation safety. The decision-making engine module dynamically adjusts the weights of costs, safety, and environmental protection objectives based on transportation stage priorities, generates a multi-level recommendation solution, and associates real-time supply chain data to implement optimized resource utilization. The execution control module uses a positioning and closed-loop feedback mechanism to control a material replacement error within 5%, and continuously improves system prediction accuracy in conjunction with dynamic calibration of a model parameter. The multidimensional data collaboration mechanism enables closed-loop optimization among environmental monitoring, material service life prediction, and supply chain scheduling.
2. According to the present invention, an intelligent collaborative network is established to substantially enhance supply chain response efficiency and risk resilience. The supply chain database integrates the material moisture resistance characteristic index table and the critical safety threshold database for hygroscopic expansion coefficients, and, in combination with the dynamic calibration mechanism for a humidity-sensitive material, rapidly generates an alternative solution in response to abrupt temperature and humidity variations and urgently executes the alternative solution through a three-step approval process. The supplier credit score system dynamically screens high-quality suppliers based on normal distribution thresholds and incorporates a closed-loop feedback mechanism using actual loss data. The self-optimization mechanism classifies and attributes a model error to respectively trigger sensor calibration, supplier data re-verification, and control parameter optimization, thereby achieving a 50% increase in monthly system iteration efficiency. The introduction of a registered dimension database together with tolerance compatibility screening logic eliminates a dimensional mismatch error between the packaging box and the material.

### DESCRIPTION OF THE EMBODIMENTS

The following clearly and completely describes the technical solutions in the embodiments of the present invention. Apparently, the described embodiments are some rather than all of the embodiments of the present invention. All other embodiments obtained by a person of ordinary skill in the art based on the embodiments of the present invention without creative efforts shall fall within the protection scope of the present invention.

The embodiments of the present invention provide an intelligent selection method and system for a packaging material based on multidimensional data analysis. Specifically, when the system is activated, an environmental sensing module uses a sensor network deployed on all six sides of a packaging box to acquire transportation environment data in real time. A vibration sensor captures three-dimensional acceleration data at a 1 kHz sampling rate and extracts an energy distribution feature in a frequency band of 0-500 Hz using fast Fourier transform. A deformation sensor monitors surface microstrain at a frequency of 100 Hz and uses a spatial interpolation algorithm to build a deformation gradient field model. A temperature-humidity sensor synchronously detects environmental parameters on each surface, and triggers an abnormal flag when a temperature difference between adjacent surfaces exceeds 3° C. or a humidity difference between adjacent surfaces exceeds 15% RH.

All sensor data is transmitted via a low-power wireless network to an edge node, where timestamp alignment and standardization are performed to generate an environmental state matrix including frequency-domain energy spectra, deformation gradients, and temperature-humidity distributions. After receiving the environmental state matrix, a data analysis module: extracts a dominant vibration frequency energy proportion feature and performs matching degree analysis with a material natural frequency database; initiates a real-time re-evaluation process of material cushioning efficiency when an energy proportion in a resonance frequency band exceeds a preset threshold; calculates a confidence interval for remaining material life combined with inventory turnover data from a supply chain database; and triggers a real-time inventory verification process if predicted life is shorter than transportation task duration obtained from a logistics management system.

A decision-making engine module dynamically adjusts target function weights based on transportation phases: in a preparation phase, a cost weight is set to 0.6, a safety weight is set to 0.3, and an environmental protection weight is set to 0.1; in a transportation phase, the safety weight is increased to 0.5; in a customs clearance stage, an environmental compliance constraint is introduced, to apply a gain coefficient of 1.2 to an environmental objective. The decision-making engine module integrates a material life prediction result with real-time supplier capacity data to generate a three-tier recommendation solution. When inventory is less than 120% of a current transportation demand, a priority of the material is automatically lowered, and a supplier database is linked to select a list of alternative suppliers with sufficient capacity and logistics timeliness that satisfies a shortest delivery cycle.

An execution control module: pre-stores a packaging box dimension tolerance standard and a structural deformation safety limit, receives material specification parameters from the decision-making engine module, matches material dimensions with a packaging box 3D model in a registered dimension database, and triggers a solution regeneration process based on actual dimension constraints if a tolerance exceeds a preset safety range; and drives a pneumatic-electromagnetic hybrid actuating mechanism to complete material replacement, receives real-time positioning accuracy feedback from a displacement sensor, initiates a PID dynamic correction mechanism if a measured position deviation exceeds 0.5 mm, and, after operation completion, acquires actual transportation data and calculates a deviation from a predicted value and feeds the deviation to the data analysis module to trigger dynamic calibration of a model parameter.

In a supplier credit score system within the supply chain database, dynamic calculation is performed based on a delivery punctuality rate weight of 0.6 and a quality inspection pass rate weight of 0.4. When a supplier's score falls below a μ−2σ value of normal distribution of historical fulfillment data, a material option of the supplier is automatically blocked. Actual loss rate data fed back by the execution control module is incorporated into scoring calculation in a next cycle at a weight of 0.2, forming a closed-loop feedback mechanism. When vibration spectrum analysis identifies that an energy value in an ±10% frequency band of a material's intrinsic frequency increases by more than 50% over a baseline within 2 seconds, the environmental sensing module sends a resonance warning signal to the data analysis module, the data analysis module retrieves transportation records with the same frequency band feature from a historical transportation case database, calculates a damage rate of a corresponding material, and sends a solution re-evaluation request to the decision-making engine module if the damage rate exceeds a safety threshold. The decision-making engine module generates a list of alternative material priorities and links the supplier database to select an alternative supplier that satisfies delivery timeliness. When a handling strategy for a humidity-sensitive material is implemented, a list of materials with hygroscopic expansion coefficients≥0.5 mm/% RH is retrieved from the supply chain database. If a humidity change rate≥15% RH/h is detected in environmental sensing data, a dedicated LSTM neural network prediction model is activated, with an input layer including a current humidity value, a change gradient, and a material hygroscopic curve feature, and an output layer predicting deformation within next two hours. If predicted deformation exceeds 80% of a structural deformation safety limit, a three-level response mechanism is triggered.

In a rapid approval process, the system automatically retrieves a customs HS code database to verify material compliance, adds an electronic fence tag to a solution involving a restricted substance, and pushes an approval request to a responsible engineer via an enterprise WeChat API, including a material parameter comparison table and a risk analysis report. The engineer uses a digital certificate to electronically sign the approval, and the system automatically records a signing timestamp and device fingerprint. Upon approval, the system immediately issues a production work order to an MES system and synchronously updates a WMS inventory state. An entire process needs to be completed within 15 minutes. If approval is not completed within a time limit, the system automatically escalates the approval to a superior and initiates a contingency plan. At the end of each quarter, a global health assessment is initiated, and a model prediction error is classified by source. When an environmental sensing error exceeds 5%, channel quality analysis and node distribution optimization are performed on the sensor network. When a material performance error exceeds 8%, supplier-provided material samples are resampled for destructive tests, and a base parameter database is updated. When an execution control error exceeds 2 mm, laser interferometer precision measurement is performed on a transmission component of a driving mechanism, and a ball screw whose wear exceeds a tolerance band is replaced. All optimization operations are performed as a closed-loop control, and each adjustment needs to undergo validation testing across three complete transportation cycles to ensure that system stability improvement satisfies a preset KPI target.

### Embodiment 2

This embodiment, based on Embodiment 1, optimizes a handling process for a humidity-sensitive material and strengthens an error tracing mechanism. Specifically, a high-precision dew point sensor is added to the environmental sensing module to monitor a condensation risk on a surface of the packaging box at a resolution of 0.1° C. When a difference between a dew point temperature and an ambient temperature is less than 2° C., a moisture-proof pretreatment instruction is triggered. An LSTM neural network in the data analysis module is upgraded to a spatiotemporal attention model, with surface deformation gradient field data added to an input layer, and the prediction time window of an output layer extended to four hours. A criterion for determining a humidity-sensitive material is tightened, and tightening means that the criterion for determining the humidity-sensitive material is tightened. Specifically, a list of materials having a hygroscopic expansion coefficient of ≥0.8 mm/% RH is dynamically updated on an hourly basis.

When a predicted deformation exceeds 60% of the safety limit, a response mechanism is triggered, with a fourth-level automatic desiccant deployment and a fifth-level cold chain logistics switching solution added. An end of a robotic arm of the execution control module is equipped with a miniature hot air gun to preheat a contact surface of the packaging box for 30 seconds before a moisture-proof material is installed, to eliminate a surface condensation film; and an adversarial sample generation technology is introduced into a calibration process to simulate an extreme humidity fluctuation scenario, thereby improving model robustness. A supplier batch defect detection functionality is added to an error attribution system. When a loss rate of materials from the same batch is abnormally high, inventory is automatically frozen and traced back to a production batch number. A rapid approval process is integrated with a blockchain-based evidence storage technology, where all electronic signatures and approval records are hashed and stored on-chain. Compared with Embodiment 1, this embodiment refines a humidity control strategy and enhances data credibility, thereby improving moisture-proof decision accuracy by 18%, reducing a rate of abnormal material loss by 27%, and increasing approval process compliance audit efficiency by 45%.

Although embodiments of the present invention have been shown and described, it can be understood that a person of ordinary skill in the art can perform various variations, modifications, substitutions, and variants on these embodiments without departing from the principle and spirit of the present invention, and the scope of the present invention is limited by the attached claims and equivalents thereof.

What is claimed is:

1. An intelligent selection system for a packaging material based on multidimensional data analysis, comprising an environmental sensing module, a data analysis module, a decision-making engine module, and an execution control module, wherein
the environmental sensing module acquires vibration spectrum, surface strain, and temperature-humidity data in real time via a sensor network deployed on a surface of the packaging box, and generates an environmental state matrix after feature extraction, wherein the sensor network comprises an automatic calibration unit, and the surface strain data is used to construct a deformation gradient field model through a spatial interpolation algorithm, for quantifying a strain distribution feature of the surface of the packaging box;
the data analysis module is internally provided with a historical transportation case database that stores performance degradation records of different materials in a transportation environment, establishes, upon receiving the environmental state matrix, a correlation model between vibration energy distribution and material performance degradation, and predicts remaining material life in conjunction with inventory turnover data from a supply chain database, and establishes a data interface with a logistics management system to obtain transportation task duration and route planning data in real time;
the decision-making engine module dynamically adjusts priorities of costs, safety, and an environmental protection objective based on transportation phases, integrates a material life prediction result with supplier capacity data, and generates a multi-level decision-making tree comprising an automatic execution solution and a contingency plan;
the execution control module: pre-stores a packaging box dimension tolerance standard, a structural deformation safety limit, and a registered dimension database; converts a selected solution into a material replacement instruction, drives an actuating mechanism to complete the operation, acquires an error between actual transportation data and a predicted value, and feeds back the error to the data analysis module to trigger dynamic model parameter calibration, and the predicted error of the model comprises an environmental sensing error, a material performance error, and an execution control error; and
the supply chain database comprises a material moisture resistance characteristic index table and a critical safety threshold database for hygroscopic expansion coefficients, records a deformation coefficient, a protection level, and an irreversible deformation threshold of each material under different humidity environments, and marks a humidity sensitivity level; and performs dynamic calculation based on a delivery punctuality rate and a quality inspection pass rate to establish a supplier credit score system, and sets a credit score threshold to a μ−2σ value of a normal distribution curve of historical fulfillment data.

2. The intelligent selection system for a packaging material based on multidimensional data analysis according to claim 1, wherein when it is determined, through vibration spectrum data analysis, that an energy value in a frequency band near a natural frequency of a material increases beyond a baseline threshold within a preset time window, the environmental sensing module sends a resonance risk warning signal to the data analysis module; and in response to the warning, the data analysis module retrieves transportation records having an energy feature in the same frequency band from the historical transportation case database, statistically analyzes damage rate data of a corresponding material, and when the damage rate exceeds a safety threshold, initiates a re-evaluation request for a current recommended solution to the decision-making engine module and generates a list of alternative material priorities.

3. The intelligent selection system for a packaging material based on multidimensional data analysis according to claim 2, wherein the data analysis module calculates a matching degree between a remaining material life prediction curve of the material and transportation task duration obtained from the logistics management system, and triggers a real-time inventory verification process in the supply chain database when the predicted remaining life is shorter than the task duration; and when a verification result indicates that an inventory level is less than 120% of a current transportation demand, the decision-making engine module automatically lowers a priority level of the material in a recommended solution, and queries the supply chain database to select a list of alternative suppliers having sufficient production capacity and logistics timeliness satisfying a shortest delivery cycle requirement, and generation of the list of alternative suppliers is matched with material types in the list of alternative material priorities.

4. The intelligent selection system for a packaging material based on multidimensional data analysis according to claim 1, wherein a recommended solution output by the decision-making engine module further comprises a material specification parameter and installation coordinate data of the packaging box, and after receiving the data, the execution control module performs matching verification between the material specification parameter and current registered dimension data of the packaging box; and when a tolerance between a material dimension and an installation position of the packaging box exceeds a preset safety range, the execution control module returns a parameter conflict warning to the decision-making engine module, triggers a regeneration process of the recommended solution based on actual dimension constraints, and preferentially selects a material option having tolerance compatibility greater than 95%.

5. The intelligent selection system for a packaging material based on multidimensional data analysis according to claim 1, wherein after installation of a new material is completed, the execution control module monitors an initial cushioning performance parameter of the new material in real time, and when a deviation between measured cushioning efficiency and a model-predicted value exceeds 5%, the execution control module sends a calibration request comprising an environmental data timestamp and a material batch code to the data analysis module; and the data analysis module extracts environmental feature data of a corresponding time window based on the request, prioritizes a coefficient iteration update for a performance degradation model of a humidity-sensitive material, and synchronizes an updated model version to the decision-making engine module.

6. The intelligent selection system for a packaging material based on multidimensional data analysis according to claim 1, wherein a credit score in the supply chain database directly influences solution generation rules of the decision-making engine module in real time, and when a credit score of a supplier falls below a threshold defined by a normal distribution curve of historical fulfillment data, the decision-making engine module automatically blocks a material option supplied by the supplier, and actual material loss rate data recorded by the execution control module is fed back to the supply chain database, so that the supply chain database dynamically adjusts a weight proportion of field performance data in a supplier quality evaluation indicator, thereby forming a closed-loop feedback mechanism between the credit score and actual material performance.

7. The intelligent selection system for a packaging material based on multidimensional data analysis according to claim 1, wherein when the environmental sensing module detects that a rate of change in temperature and humidity exceeds the safety-critical threshold of the hygroscopic expansion coefficient of the material, the data analysis module immediately initiates deformation prediction calculation and evaluates a risk level with reference to a structural deformation safety limit of the packaging box; if a predicted deformation exceeds 80% of a safety limit, the decision-making engine module terminates current solution execution and transmits an emergency reinforcement command to the execution control module, and based on a material moisture resistance characteristic index table stored in the supply chain database, an alternative material recommendation list is generated, and a rapid approval process is initiated, wherein the rapid approval process comprises three steps: automatic compliance verification, electronic signature confirmation by a responsible person, and automatic issuance of an execution command.

8. The intelligent selection system for a packaging material based on multidimensional data analysis according to claim 1, wherein the data analysis module periodically performs classification and attribution on the predicted errors of the model, and when the environmental sensing error exceeds a preset tolerance for three consecutive times, a sensor calibration unit of the environmental sensing module is triggered to perform accuracy verification; when cumulative material performance errors exceed a predefined threshold, a supplier historical data review process is initiated, and a credit score is recalculated; and when an execution control error persists, control parameter optimization rules of the actuating mechanism are dynamically adjusted until actual operating accuracy reaches a preset standard.

9. A selection method proposed based on the intelligent selection system for a packaging material based on multidimensional data analysis according to claim 1, wherein the environmental sensing module uses the sensor network on the surface of the packaging box to acquire the vibration spectrum, surface strain, and temperature and humidity data in real time, to generate the environmental state matrix; the data analysis module predicts the remaining material life combined with the historical transportation case database and the supply chain database and communicates with the logistics management system to obtain transportation task information; the decision-making engine module dynamically adjusts target priorities based on transportation stages and integrates material life prediction and supplier capacity data to generate the multi-level decision-making tree; the execution control module executes material replacement based on a selected solution and acquires the error between the actual transportation data and the predicted value, and feeds back the error to the data analysis module, triggering dynamic calibration of a model parameter; and the supply chain database comprises the material moisture resistance characteristic index table and the critical safety threshold database for hygroscopic expansion coefficients, records the deformation coefficient, the protection level, and the irreversible deformation threshold of each material under different humidity environments, and establishes the supplier credit score system based on the delivery punctuality rate and the quality inspection pass rate.
